(19) United States
(12) Patent Application Publication
(10) Pub. No.: US 2019/0231201 A1
(43) Pub. Date: Aug. 1, 2019

(54) PHOTOELECTRIC SENSOR, PHOTOELECTRIC SENSOR MODULE, AND BIOLOGICAL INFORMATION MEASUREMENT APPARATUS

(71) Applicant: SEIKO EPSON CORPORATION, Tokyo (JP)

(72) Inventor: Kazuyuki KANO, Suwa-shi (JP)

(73) Assignee: SEIKO EPSON CORPORATION, Tokyo (JP)

(21) Appl. No.: 16/261,943

(22) Filed: Jan. 30, 2019

(30) Foreign Application Priority Data
Jan. 31, 2018 (JP) 2018-015683

(51) Int. Cl. A61B 5/024; H01L 27/32

(52) U.S. Cl. CPC A61B 5/02427; A61B 2562/166; A61B 2562/0233; H01L 27/3227

(57) ABSTRACT

A photoelectric sensor includes a silicon substrate, a light emitter that emits first light, a light receiver that receives second light, a light emitter terminal that supplies the light emitter with electric power, and a light receiver terminal to which the light receiver outputs a signal, and the light emitter, the light receiver, the light emitter terminal, and the light receiver terminal are provided on the silicon substrate.

### CROSS-REFERENCES TO RELATED APPLICATIONS

This application claims priority to Japanese Patent Application No. 2018-015683, filed on Jan. 31, 2018, the entirety of which is herein incorporated by reference.

### BACKGROUND

### 1. Technical Field

The present invention relates to a photoelectric sensor, a photoelectric sensor module, and a biological information measurement apparatus.

### 2. Related Art

There has been a known measurement apparatus capable of measuring biological information. As an example of the measurement apparatus, there is a known biological information measurement apparatus including a sensor unit formed of a light emitter that emits light to a target object and a light receiver that receives light from the target object and serving as a biological information measurement module (see JP-A-2016-123715, for example).

In the biological information measurement apparatus described in JP-A-2016-123715, the light emitter and the light receiver are mounted on a substrate. Out of the two components, the light emitter is achieved, for example, by a light emitting diode (LED) or any other light emitting device, and the light receiver is achieved, for example, by a photodiode or any other light receiving device. Further, the light emitter is provided with a dome-shaped lens, and the lens collects the light from the LED chip encapsulated with a light transmissive resin in the light emitter.

Considering a pulsimeter as the measurement apparatus, the light emitted from the light emitter travels through the interior of a subject that is the target object and is diffused or scattered in the epidermis, dermis, subcutaneous tissue, and other body sites. The light then reaches a blood vessel (site to be detected) and is reflected off the blood vessel. In this process, since part of the light is absorbed by the blood vessel, the pulse affects and changes the proportion of the light absorbed by the blood vessel, and the amount of reflected light changes accordingly. Detection of a change in the amount of reflected light received with the light receiver allows measurement of the pulse rate and other factors that are each biological information.

In the biological information measurement apparatus described in JP-A-2016-123715, however, the light emitter and the light receiver are mounted on the substrate. Therefore, for example, a packaged LED chip is employed as the light emitter, and/or a packaged photodiode (PD) or phototransistor (PT) chip is employed as the light receiver in some cases. In any of the cases described above, the sensor unit tends to be thick in the light exiting direction.

In recent years, a wearable apparatus including a sensor that measures biological information has been increasingly familiar. Since such a wearable apparatus is worn by a user when used, the wearable apparatus is desirably so sized as not to hinder the user's action. When the thickness of the sensor increases as described above, however, the thickness of a housing that accommodates the sensor tends to increase, resulting in a problem of poor usability felt by the user.

In view of the circumstances described above, a sensor that allows a small thickness thereof has been desired.

### SUMMARY

An advantage of some aspects of the invention is to provide a photoelectric sensor, a photoelectric sensor module, and a biological information measurement apparatus that allow a small thickness thereof.

A photoelectric sensor according to a first aspect of the invention includes a silicon substrate, a light emitter that emits first light, a light receiver that receives second light, a light emitter terminal that supplies the light emitter with electric power, and a light receiver terminal to which the light receiver outputs a signal, and the light emitter, the light receiver, the light emitter terminal, and the light receiver terminal are provided on the silicon substrate.

In a case where the photoelectric sensor is employed as a photoelectric sensor that detects biological information associated with a living body, the first light emitted from the light emitter is light with which the living body is irradiated, and the second light received with the light receiver is light received via the living body and containing information, for example, the pulse wave or body motion.

According to the configuration described above, the photoelectric sensor has the configuration in which the light emitter, the light emitter terminal, the light receiver, and the light receiver terminal are provided on the silicon substrate. The light emitter and the light receiver can therefore be configured in the form of a non-packaged bare chip on the silicon substrate. The thickness of the photoelectric sensor can be reduced as compared with a case where a packaged LED chip and a packaged photodiode are mounted on the silicon substrate. Further, since the light emitter and the light receiver are configured on the silicon substrate, no packaged LED chip or photodiode needs to be attached to the silicon substrate, for example, via a bump. The dimension from the light emitter and the light receiver to the silicon substrate can therefore be reduced. The thickness of the photoelectric sensor can therefore be reduced, whereby a thinner photoelectric sensor can be achieved. Moreover, since not only can the number of constituent parts provided in the photoelectric sensor be reduced, but the steps of manufacturing the photoelectric sensor can be simplified, whereby the cost of the photoelectric sensor can be reduced.

In general, in a photoelectric sensor, a smaller distance between the light emitter and the light receiver results in a greater amount of light received with the light receiver and hence higher accuracy of detection of a detection target.

On the other hand, to mount a packaged LED chip and a packaged photodiode on the silicon substrate, a gap between the packaged LED chip and the packaged photodiode is required for the mounting operation.

In contrast, formation of the light emitter and the light receiver on the silicon substrate allows reduction in the distance between the light emitter and the light receiver as compared with the case described above. The amount of light received with the light receiver can therefore be readily increased, whereby the sensitivity of the detection of the target detected with the photoelectric sensor can be increased.

In the first aspect described above, it is preferable that the light emitter and the light receiver are provided on the same surface of the silicon substrate.

The situation in which the light emitter and the light receiver are provided on the same surface of the silicon substrate indicates that the components described above are disposed on a single surface of the silicon substrate. The light receiver has a p-type area and n-type areas provided in the silicon substrate, and it is assumed that the light emitter and the light receiver are provided on the same surface, including the case described above in which part of the components is provided in the silicon substrate.

According to the configuration described above, the thickness of the photoelectric sensor can be further reduced as compared with a case where the light emitter and the light receiver are provided on the opposite surfaces of the silicon substrate, whereby a further thinner photoelectric sensor can be achieved.

In the first aspect described above, it is preferable that the photoelectric sensor further includes a light blocker provided on the silicon substrate and between the light emitter and the light receiver.

According to the configuration described above, the light blocker prevents the light emitted from the light emitter from being directly incident on the light receiver. The accuracy of the detection of the target detected with the photoelectric sensor can therefore be increased.

In the first aspect described above, it is preferable that the light emitter is an organic EL device.

According to the configuration described above, an organic EL device and hence a light emitter that can emit light can be formed by formation of a cathode layer, an organic layer, and an anode layer on the silicon substrate. The light emitter can therefore be readily formed on the silicon substrate.

In the first aspect described above, it is preferable that the light emitter has a plurality of light emitting areas, and that the light emitter terminal is provided in accordance with the plurality of light emitting areas.

According to the configuration described above, the plurality of light emitting areas can be separately caused to emit light, whereby the versatility of the photoelectric sensor can be expanded.

For example, adjusting the number of light emitting areas to be caused to emit light can adjust the mount of light emitted from the light emitter. In addition, for example, providing the light emitting areas with different color filter layers or polarizing layers allows light having a wavelength or polarization according to the application to be emitted from the light emitter.

In the first aspect described above, it is preferable that the light emitter is so disposed as to surround the light receiver.

According to the configuration described above, for example, the light emitted from the light emitter can be readily incident on the light receiver via the body of the target object (living body, for example). The amount of light received with the light receiver can therefore be readily increased. In a case where the photoelectric sensor detects, for example, biological information, the accuracy of the detection of the biological information can be increased.

In the first aspect described above, it is preferable that the light emitter emits green light.

In the case of a photoelectric sensor that detects biological information relating to the blood, such as the blood flow and the pulse wave, it is preferable to use green light that has higher in-blood hemoglobin absorptance and is less affected by ambient light than light having the other colors as the light introduced into the living body.

Causing the light emitter to emit green light can therefore configure the photoelectric sensor according to the first aspect described above as the photoelectric sensor that detects biological information relating to the blood.

In the first aspect described above, it is preferable that the light receiver includes an angle limiting layer that limits an angle of incidence of the light incident on the light receiver.

According to the configuration described above, the angle limiting layer can prevent the light that does not travel through a target irradiated with the light emitted from the light emitter but externally travels toward the light receiver or ambient light from being incident on the light receiver. False detection of the detection target can therefore be avoided, whereby the detection accuracy of the photoelectric sensor can be increased.

In the first aspect described above, it is preferable that the light receiver includes an optical thin film layer that transmits light that belongs to a predetermined band out of wavelength bands of light incident on the light receiver.

In other words, in the first aspect described above, it is preferable that the light receiver includes an optical thin film layer that prevents transmission of light that belongs to the wavelength band other than the wavelength band to which the light received with the light receiver belongs.

The light that belongs to the predetermined band can, for example, be light that belongs to a band containing the wavelength of the first light emitted from the light emitter.

According to the configuration described above, the optical thin film layer can prevent the light receiver from receiving light that belongs to a wavelength band different from the wavelength band to which the light that the light receiver should receive belongs. The influence of noise can therefore be reduced, whereby the detection sensitivity of the photoelectric sensor can be increased.

In the first aspect described above, it is preferable that the photoelectric sensor further includes a translucent member that covers the light emitter and the light receiver.

According to the configuration described above, the translucent member can prevent the light emitter and the light receiver from being directly exposed to the outside, whereby the light emitter and the light receiver can be protected.

A photoelectric sensor module according to a second aspect of the invention includes the photoelectric sensor described above, a sensor control section that controls the photoelectric sensor, and a printed circuit board on which the photoelectric sensor and the sensor control section are located, and the sensor control section at least turns on the light emitter and/or processes the signal from the light receiver.

According to the configuration described above, not only can the same effects as those provided by the photoelectric sensor according to the first aspect described above be provided but the photoelectric sensor module can have a compact configuration because the sensor control section and the photoelectric sensor are located on the printed circuit board.

The sensor control section has the function as a light emission controller that controls turn-on operation of the light emitter and the function as a signal processor that processes the signal from the light receiver. Therefore, as long as the sensor control section has the functions as the light emission controller and the signal processor, the configuration of the photoelectric sensor module can be simplified as compared with a case where circuits according to the functions are separately provided.

A biological information measurement apparatus according to a third aspect of the invention includes the photoelectric sensor module described above, a circuit substrate that derives biological information based on a signal outputted from the photoelectric sensor module, and a connection member that electrically connects the printed circuit board to the circuit substrate.

Examples of the biological information may include the pulse rate derived based on a pulse-wave signal representing the pulse wave detected with the photoelectric sensor module.

According to the configuration described above, the same effects as those provided by the photoelectric sensor module according to the second aspect described above can be provided. Further, since print circuit board in the photoelectric sensor module is electrically connected to the circuit substrate, the circuit substrate can reliably derive the biological information based on the result of the detection performed by the photoelectric sensor module. Since the photoelectric sensor module connected to the circuit substrate via the connection member can be changed to change biological information derivable by the circuit substrate, the versatility of the biological information measurement apparatus can be increased.

### BRIEF DESCRIPTION OF THE DRAWINGS

The invention will be described with reference to the accompanying drawings, wherein like numbers reference like elements.

FIG. 1 is a diagrammatic view showing an example of how to use a biological information measurement apparatus according to a first embodiment of the invention.

FIG. 2 is a front view showing the biological information measurement apparatus in the first embodiment.

FIG. 3 shows a rear surface section of a housing in the first embodiment.

FIG. 4 is a block diagram showing the configuration of the biological information measurement apparatus in the first embodiment.

FIG. 5 shows a photoelectric sensor module in the first embodiment viewed from a light output side.

FIG. 6 shows a photoelectric sensor in the first embodiment viewed from the light output side.

FIG. 7 is a cross-sectional view showing the photoelectric sensor in the first embodiment.

FIG. 8 is a flowchart showing a measurement control process in the first embodiment.

FIG. 9 is a flowchart showing the steps of manufacturing the photoelectric sensor in the first embodiment.

FIG. 10 shows a photoelectric sensor of a photoelectric sensor module provided in a biological information measurement apparatus according to a second embodiment of the invention viewed from the light output side.

FIG. 11 shows a photoelectric sensor according to a first variation viewed from the light output side.

FIG. 12 shows a photoelectric sensor according to a second variation viewed from the light output side.

### DESCRIPTION OF EXEMPLARY EMBODIMENTS

### First Embodiment

A first embodiment of the invention will be described below with reference to the drawings.

Schematic configuration of biological information measurement apparatus

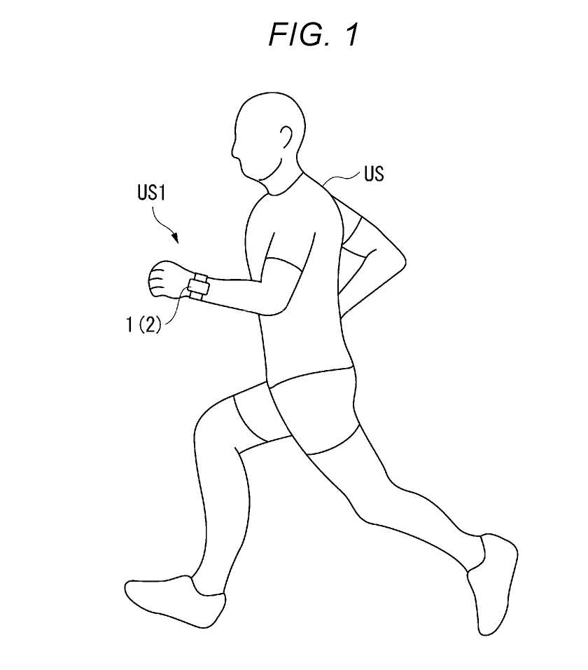

FIG. 1 is a diagrammatic view showing an example of how to use a biological information measurement apparatus 1 according to the present embodiment.

The biological information measurement apparatus 1 according to the present embodiment (hereinafter abbreviated to measurement apparatus 1 in some cases) is a wearable apparatus that is worn on the body of a user US when used, as shown in FIG. 1, and measures biological information associated with the user US. Specifically, the measurement apparatus 1 is worn on an apparatus worn site US1, such as a wrist of the user US, when used, detects the pulse wave of the user US as the biological information, and measures the pulse rate, which is also the biological information.

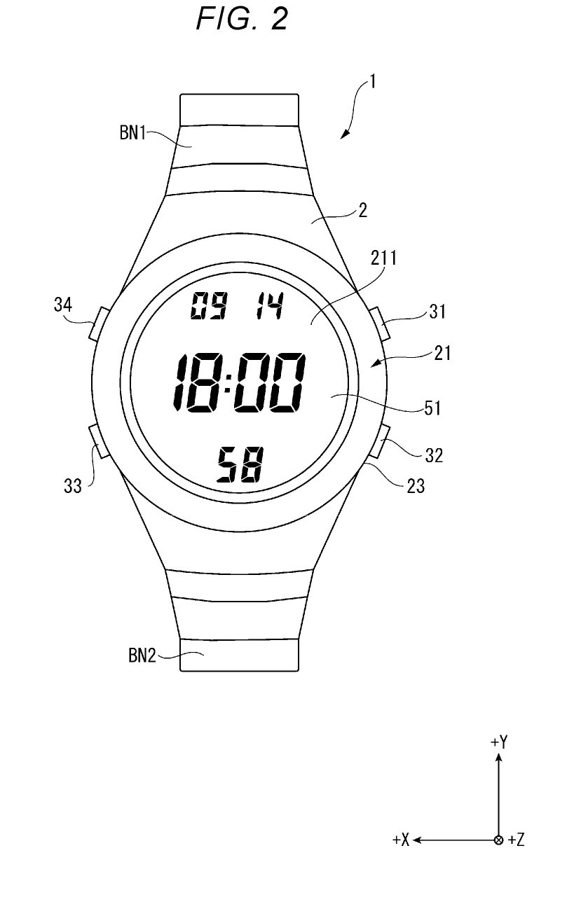

FIG. 2 shows the exterior appearance of the measurement apparatus 1.

The measurement apparatus 1 includes a housing 2 and bands BN1 and BN2 attached to the housing 2, as shown in FIG. 2.

In FIG. 2 and the following figures, the direction from a front surface section 21 of the housing 2 toward a rear surface section 22 thereof is called a direction +Z. The two directions perpendicular to the direction +Z are called directions +Y and +X, and it is assumed that the direction +X is the 9-o'clock direction and the direction +Y is the 12-o'clock direction when the measurement apparatus 1 is viewed from a position facing the front surface section 21. Although not shown, the direction opposite the direction +Z is called a direction −Z. The same holds true for directions −X and −Y.

The bands BN1 and BN2 are connected to the ±Y-direction ends of the housing 2. The band BN1 extends toward the +Y-direction side with respect to the housing 2, and the band BN2 extends toward the −Y-direction side with respect to the housing 2. When the bands BN1 and BN2 are linked to each other via a buckle (not shown), the housing 2 is worn on the apparatus worn site US1 described above. The bands BN1 and BN2 may instead be formed integrally with the housing 2.

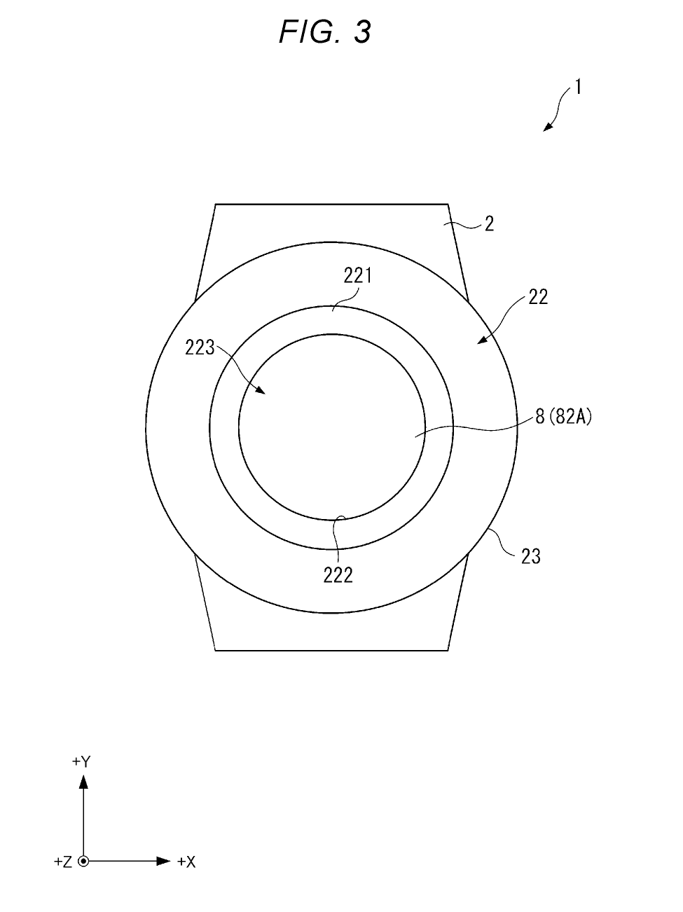

The housing 2 has the front surface section 21, the rear surface section 22 (see FIG. 3), and a side surface section 23.

The front surface section 21 is a portion of the housing 2 that is the portion located on the −Z-direction side, and a display section 51, which forms a notification section 5, which will be described later, is provided roughly at the center of the front surface section 21. That is, the front surface section 21 is a portion that allows the user US who wears the measurement apparatus 1 via the housing 2 can visually recognize a content displayed in the display section 51. The display section 51 is covered with a circular cover 211. In the present embodiment, the cover 211 is a translucent member and is, for example, a windshield glass plate. The direction of a normal to the display surface of the display section 51 is parallel to the direction −Z.

The side surface section 23 is an annular section formed along the circumferential direction around the direction +Z direction and connects the front surface section 21 and the rear surface section 22 to each other. Buttons 31 and 32, which form an operation section 3, which will be described later, are disposed in a −X-direction-side area of the side surface section 23, and buttons 33 and 34, which also form the operation section 3, are disposed in a +X-direction-side area of the side surface section 23. The buttons 31 to 34 protrude and retract with respect to the housing 2.

FIG. 3 shows the rear surface section 22 of the housing 2. In FIG. 3, the buttons 31 to 34 are omitted.

The rear surface section 22 is not only a portion of the housing 2 that is the portion located on the +Z-direction side but a portion of the housing 2 that faces and comes into contact with the apparatus worn site US1.

A roughly annular protrusion 221 is formed at the center of the rear surface section 22. An opening 222 is formed at the center of the protrusion 221, and the inner area of the opening 222 forms a placement section 223, where a photoelectric sensor 82A, which forms a photoelectric sensor module 8, which will be described later, is placed.

### Detailed Configuration of Biological Information Measurement Apparatus

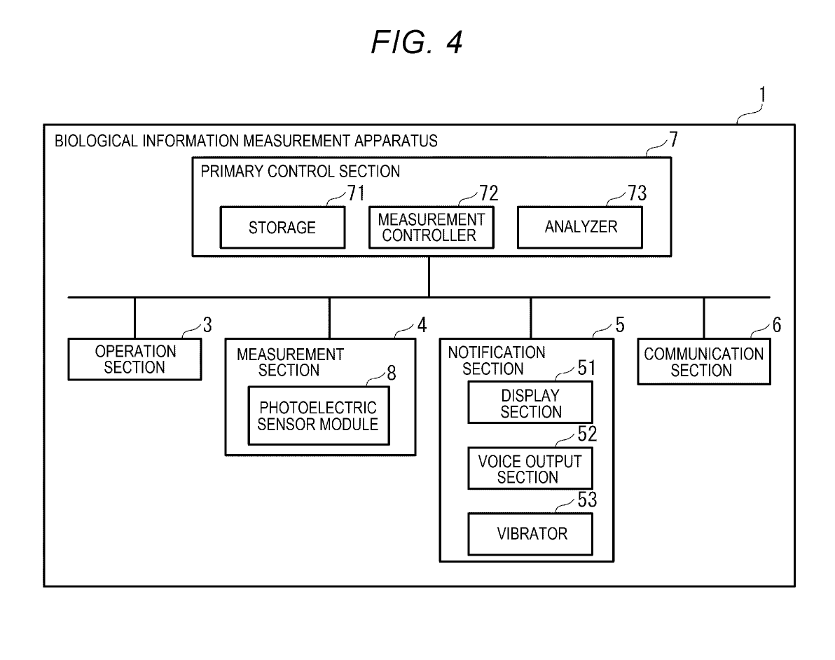

FIG. 4 is a block diagram showing the configuration of the measurement apparatus 1.

The measurement apparatus 1 includes not only the housing 2 but the operation section 3, a measurement section 4, the notification section 5, a communication section 6, a primary control section 7, which are provided in the housing 2, as shown in FIG. 4.

The operation section 3 includes the buttons 31 to 34 described above and outputs an operation signal according to an input performed by pushing any of the buttons 31 to 34 to the primary control section 7.

The measurement section 4 detects biological information and outputs the result of the detection to the primary control section 7. The measurement section 4 includes the photoelectric sensor module 8, which outputs a pulse-wave signal representing the pulse wave, which is the biological information, to the primary control section 7. The configuration of the photoelectric sensor module 8 will be described later in detail.

The notification section 5 notifies the user of a variety of pieces of information under the control of the primary control section 7. The notification section 5 includes the display section 51, a voice output section 52, and a vibrator 53.

The display section 51 has a variety of display panels, such as a liquid crystal display panel and an electronic paper display panel and displays information inputted from the primary control section 7. The display section 51 displays, for example, the pulse rate detected and analyzed by the measurement section 4.

The voice output section 52 outputs voice according to a voice signal inputted from the primary control section 7.

The vibrator 53 includes a motor that acts under the control of the primary control section 7, and the vibrator 53 notifies the user, for example, of warning in the form of vibration produced by the driven motor.

The communication section 6 is a communication module that transmits detected and measured biological information to an external apparatus and outputs information received from the external apparatus to the primary control section 7. In the present embodiment, the communication section 6 wirelessly communicates with the external apparatus by using a short-range wireless communication method and may instead communicate with the external apparatus via a cradle or any other relay device or a cable. The communication section 6 may still instead communicate with the external apparatus over a network.

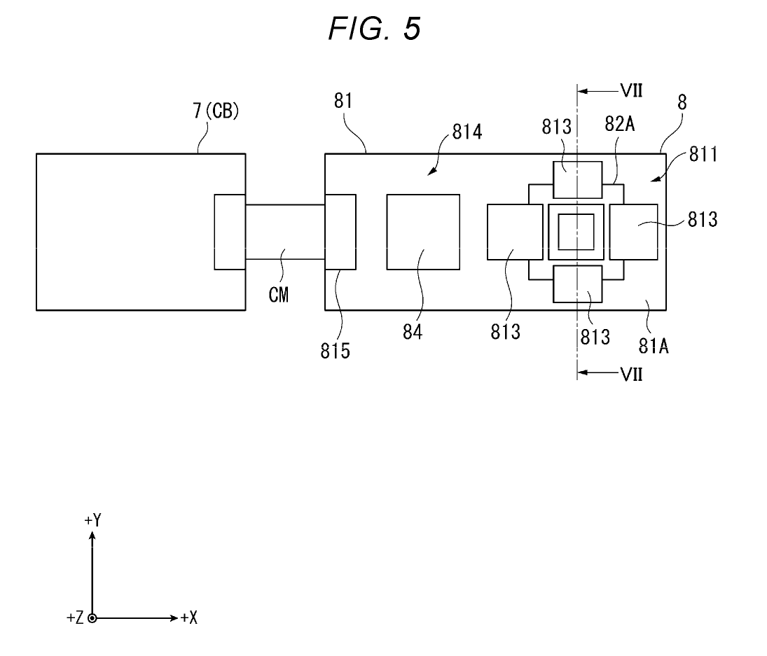

The primary control section 7 is formed of a circuit substrate CB (see FIG. 5) including a computation circuit and a flash memory and is electrically connected to the operation section 3, the measurement section 4, the notification section 5, and the communication section 6. The primary control section 7 controls the action of the entire measurement apparatus 1 autonomously or in accordance with the operation signal inputted from the operation section 3 described above. In addition to the above, the primary control section 7 controls the action of the photoelectric sensor module 8 to analyze the pulse-wave signal inputted from the photoelectric sensor module 8.

The thus configured primary control section 7 includes a storage 71, which is formed of the flash memory described above, and a measurement controller 72 and an analyzer 73, which are formed of the computation circuit described above that executes a program stored in the storage 71.

The storage 71 stores a variety of programs and data necessary for the action of the measurement apparatus 1. The storage 71 further stores the pulse-wave signal inputted from the photoelectric sensor module 8 and the pulse rate analyzed by the analyzer 73.

The measurement controller 72 controls the action of the photoelectric sensor module 8 to cause the photoelectric sensor module 8 to detect the pulse wave. A measurement control process carried out by the measurement controller 72 will be described later.

The analyzer 73 analyzes the pulse-wave signal inputted from the photoelectric sensor module 8 to calculate the pulse rate. Specifically, the analyzer 73 performs frequency analysis, such as fast Fourier transform (FFT), on the pulse-wave signal, extracts the frequency of the pulse from the result of the analysis, and calculates the pulse rate based on the extracted frequency. The analyzer 73 may calculate the pulse rate by using another approach.

### Configuration of Photoelectric Sensor Module

FIG. 5 shows the photoelectric sensor module 8 viewed from the light output side. In other words, FIG. 5 shows the photoelectric sensor module 8 viewed from the side facing the interior of the housing 2. In FIG. 5, the photoelectric sensor module 8 and the circuit substrate CB, which forms the primary control section 7, is disposed along the direction +X for ease of description, but the photoelectric sensor module 8 and the circuit substrate CB may instead be so disposed as to overlap with each other in the direction +Z via a connection member CM.

The photoelectric sensor module 8 outputs light to the body of the user US and detects biological information based on a change in the amount of light reflected off the body (reflected light) or otherwise incident on the photoelectric sensor module 8. In detail, the photoelectric sensor module 8 detects the pulse wave, which is the biological information, in the form of a change in the amount of received light and outputs the pulse-wave signal representing the detected pulse wave to the primary control section 7.

The thus configured photoelectric sensor module 8 includes a printed circuit board 81 and the photoelectric sensor 82A and a sensor control section 84, which are provided on the printed circuit board 81, as shown in FIG. 5.

### Configuration of Printed Circuit Board

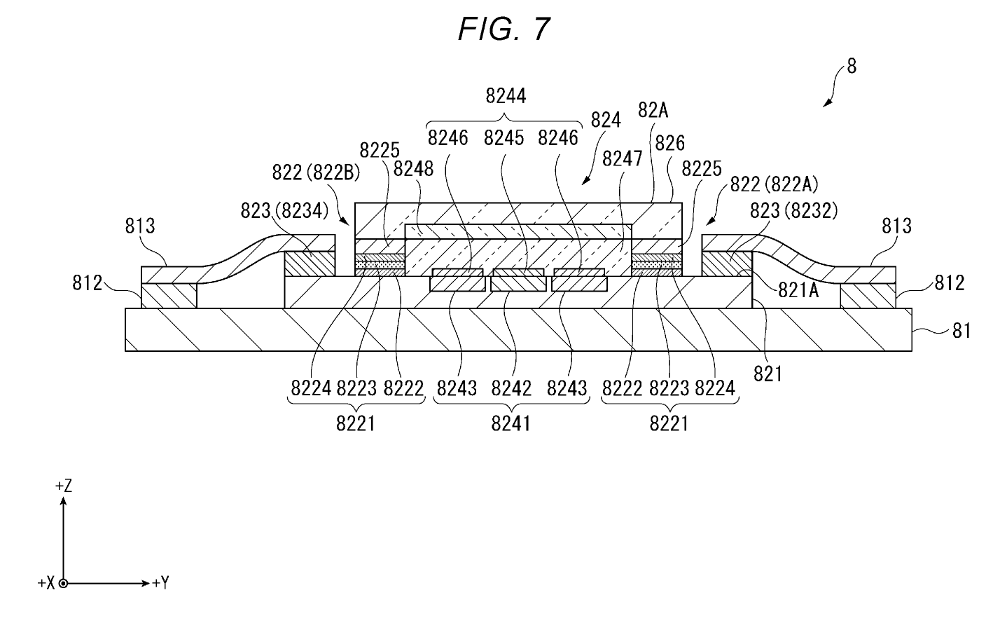

The printed circuit board 81 is a rigid substrate (board-shaped hard substrate) having a roughly oblong shape. The printed circuit board 81 has the following components mounted on a mount surface 81A, which is the +Z-direction-side surface: a sensor placement section 811; a plurality of substrate-side electrodes 812 (see FIG. 7); a plurality of flexible printed circuits (FPCs) 813; a circuit placement section 814; and a connector 815.

The sensor placement section 811 is a portion where the photoelectric sensor 82A is placed and which is located in a +X-direction-side portion of the printed circuit board 81.

The plurality of substrate-side electrodes 812 are provided around the sensor placement section 811.

The plurality of FPCs 813 connect the substrate-side electrodes 812 to light emitter terminals 823 and light receiver terminals 825, which will be described later in the description of the photoelectric sensor 82A.

The circuit placement section 814 is a portion where the sensor control section 84 is placed and which is located in a −X-direction-side portion of the printed circuit board 81 relative to the sensor placement section 811.

The connector 815 is located in a −X-direction-side end portion of the mount surface 81A. The printed circuit board 81 is electrically connected to the circuit substrate CB, which forms the primary control section 7, via the connection member CM, which is connected to the connector 815. The connection member CM can, for example, be an FPC or a wire harness.

### Configuration of Sensor Control Section

The sensor control section 84 will be described before the photoelectric sensor 82A.

The sensor control section 84 is formed of an integrated circuit placed in the circuit placement section 814. The sensor control section 84 not only controls the action of the photoelectric sensor 82A but processes the pulse-wave signal inputted from the photoelectric sensor 82A via the printed circuit board 81 and outputs the processed pulse-wave signal to the primary control section 7 via the connector 815.

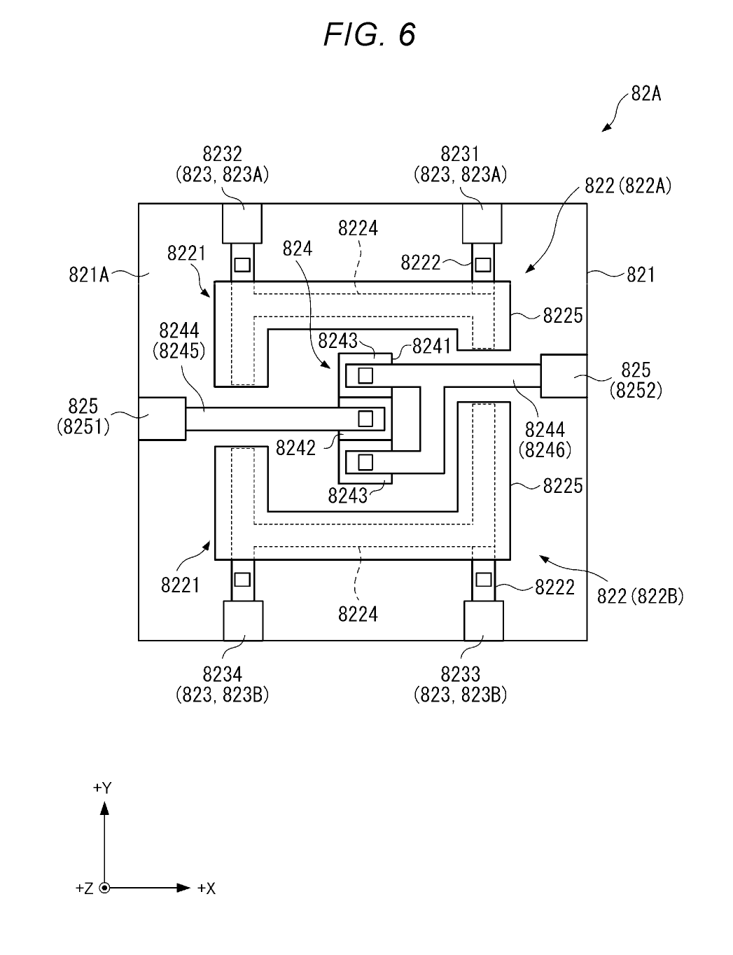

Specifically, the sensor control section 84 has the function as a light emission controller that turns on a light emitter 822 in accordance with a control signal inputted from the measurement controller 72 of the primary control section 7 connected to the printed circuit board 81 via the connection member CM. In detail, the sensor control section 84 controls the light emission state of each of a first light emitting area 822A and a second light emitting area 822B (see FIGS. 6 and 7), which form the light emitter 822 of the photoelectric sensor 82A, in accordance with the inputted control signal.

The sensor control section 84 further has the function as a signal processor that performs amplification, filtering, A/D conversion, and other types of processing on the pulse-wave signal inputted from the photoelectric sensor 82A. That is, the sensor control section 84 has the function of an analog front end (AFE). The thus processed pulse-wave signal is outputted to the primary control section 7.

### Configuration of Photoelectric Sensor

FIG. 6 shows the photoelectric sensor 82A viewed from the light output side (+Z-direction side). In FIG. 6, part of the components of the light emitter 822 and a light receiver 824 is omitted.

The photoelectric sensor 82A is a reflective photoelectric sensor that outputs light that is green light, receives light outputted from the user's body, and outputs a detection signal according to the amount of received light as the pulse-wave signal (reflective pulse-wave sensor). The photoelectric sensor 82A includes a silicon substrate 821, the light emitter 822, the light emitter terminal 823, the light receiver 824, the light receiver terminals 825, and a translucent member 826, as shown in FIG. 6.

### Configuration of Silicon Substrate

The silicon substrate 821 is a semiconductor substrate and is formed in a roughly square shape when the photoelectric sensor 82A is viewed from the light output side. A surface 821A of the silicon substrate 821, which is the surface oriented in the direction +Z (+Z-direction-side surface 821A) is a surface on which the light emitter 822, the light emitter terminals 823, the light receiver 824, and the light receiver terminals 825 are provided (surface on which the components described above are formed).

The light receiver 824 has a p-type area 8242 and n-type areas 8243, which will be described later, are provided in the silicon substrate 821, and it is assumed that the light emitter 822, the light emitter terminals 823, the light receiver 824, and the light receiver terminals 825 are provided on the same surface 821A, including the case described above in which part of the p-type area 8242 and the n-type areas 8243 is provided in the silicon substrate 821.

The step of forming the light emitter 822 and the light receiver 824 are formed on the thus configured silicon substrate 821 will be described later in detail.

### Configuration of Light Receiver

FIG. 7 is a cross-sectional view showing the photoelectric sensor module 8. In detail, FIG. 7 shows the cross section of the photoelectric sensor 82A taken along the line VII-VII in FIG. 5.

The light receiver 824 will next be described.

The light receiver 824 receives the light reflected off the body (blood vessel inexact sense) of the user US (second light) and outputs a signal according to a change in the amount of the reflected light as the pulse-wave signal. The light receiver 824 is formed roughly at the center of the silicon substrate 821.

The thus configured light receiver 824 includes not only a light receiving layer 8241 and a light receiving electrode layer 8244, as shown in FIG. 6, but an angle limiting layer 8247 and an optical thin film layer 8248, as shown in FIG. 7.

The light receiving layer 8241 has one p-type area 8242 (p layer) and two n-type areas 8243 (n layers), which sandwich the one p-type area 8242 in the direction +Y. That is, the light receiver 824 is a pn-junction-type photodiode. The area where the p-type area 8242 and the n-type areas 8243 are located is a light receiving area of the light receiver 824, and the direction of a normal to the light receiving area is parallel to the direction +Z.

The light receiving electrode layer 8244 includes a p electrode layer 8245, which covers a +Z-direction-side portion of the p-type area 8242 and is connected to the p-type area 8242, and an n electrode layer 8246, which covers +Z-direction-side portions of the two n-type areas 8243 and is connected to the two n-type areas 8243.

The p electrode layer 8245 extends from the p-type area 8242 in the direction −X and is connected to a p terminal 8251, which is one of the light receiver terminals 825.

The n electrode layer 8246 extends from the n-type areas 8243 in the direction +X and is connected to an n terminal 8252, which is one of the light receiver terminals 825.

The angle limiting layer 8247 is so provided as to cover the +Z-direction side light of the receiving layer 8241, as shown in FIG. 7. The angle limiting layer 8247 is formed of a plurality of columnar portions arranged at predetermined intervals along the direction of a normal to the light receiving area (direction +Z), and the angle limiting layer 8247 transmits light incident at angles of incidence smaller than a predetermined angle and prevents transmission of light incident at angles of incidence greater than or equal to the predetermined angle. The columnar portions are arranged at intervals set in accordance with the wavelength of the incident light and can be so arranged that d2/λR≥2 is satisfied, where λ represents the wavelength of the incident light, R represents the height of the columnar portions, and d represents the interval between the columnar portions in the cross-sectional view viewed in the direction perpendicular to the direction of a normal to the light receiving area.

The optical thin film layer 8248 is so provided as to cover the +Z-direction side of the angle limiting layer 8247. The optical thin film layer 8248 is a filter layer that transmits light that belongs to a wavelength band (predetermined band) to which the light emitted by the light emitter 822 belongs, out of the wavelength bands of the light traveling toward the light receiver 824, and prevents transmission of light that belongs to the other wavelength band.

### Configuration of Light Receiver Terminals

The light receiver terminals 825 include the p terminal 8251, which is located in a −X-direction-side edge portion of the silicon substrate 821, and the n terminal 8252, which is located in a +X-direction-side edge portion of the silicon substrate 821, as shown in FIG. 6.

The p terminal 8251 is not only a terminal connected to the p electrode 8245 but the anode of the pn-junction-type photodiode.

The n terminal 8252 is not only a terminal connected to the n electrodes 8246 but the cathode of the pn-junction-type photodiode.

The light receiver 824 is connected to the printed circuit board 81 via the p terminal 8251, the n terminal 8252, and the FPCs 813.

When light is incident on one of the junction surfaces between the p-type area 8242 and the two n-type areas 8243 of the light receiver 824, the photovoltaic effect causes current according to the intensity of the light to be outputted to the n terminal 8252 via the n electrode layer 8246. The current outputted to the n terminal 8252 is outputted to the printed circuit board 81 via the FPSs 813. Although will be described later, the current outputted to the printed circuit board 81 is inputted to the sensor control section 84 (see FIG. 5) mounted on the printed circuit board 81.

### Configuration of Light Emitter

The light emitter 822 is an organic EL device formed on the silicon substrate 821 and emits light that is green light (first light) to the body of the user US. In the present embodiment, the light emitter 822 has the first light emitting area 822A, which is located on the +Y-direction side and formed in a roughly U-letter shape that opens toward the −Y-direction side, and the second light emitting area 822B, which is located on the −Y-direction side and formed in a roughly U-letter shape that opens toward the +Y-direction side, as shown in FIG. 6. That is, the light emitter 822 has the first light emitting area 822A and the second light emitting area 822B, which are a plurality of light emitting areas that are divided areas. The first light emitting area 822A and the second light emitting area 822B are so provided as to surround the light receiver 824 with a predetermined gap between the light emitting areas and the light receiver.

The first light emitting area 822A and the second light emitting area 822B each have the same layer structure.

Specifically, the light emitting areas 822A and 822B each have a light emitting layer 8221 and a filer layer 8225, as shown in FIG. 7.

The light emitting layer 8221 has a cathode layer 8222, an organic layer 8223, and an anode layer 8224. That is, the light emitting layer 8221 is so configured that the cathode layer 8222, the organic layer 8223, and the anode layer 8224 are layered on each other sequentially from the side facing the silicon substrate 821, and the organic layer 8223 is sandwiched between the cathode layer 8222 and the anode layer 8224 in the direction +Z.

The cathode layer 8222 in the first light emitting area 822A is connected to a terminal 8231 (see FIG. 6), which is one of first light emitting area terminals 823A, which will be described later, and the anode layer 8224 in the first light emitting area 822A is connected to a terminal 8232 (see FIG. 6), which is the other one of the first light emitting area terminals 823A.

The cathode layer 8222 in the second light emitting area 822B is connected to a terminal 8233 (see FIG. 6), which is one of second light emitting area terminals 823B, which will be described later, and the anode layer 8224 in the second light emitting area 822B is connected to a terminal 8234 (see FIG. 6), which is the other one of the second light emitting area terminals 823B.

At least the anode layers 8224, which need to transmit the light outputted from the organic layers 8223, are each a transparent electrode layer that can transmit light.

The filter layers 8225 are so formed as to cover the light emitting layers 8221. The filter layers 8225 transmit the light that the photoelectric sensor 82A should output (green light, for example) and block the other color light. The reason why the filter layers 8225 are provided is that the light emitting layers 8221 according to the present embodiment are configured to output white light, and if the light emitting layers 8221 themselves can output green light, the filter layers 8225 can be omitted. Instead, in place of the filter layers 8225, wavelength conversion layers that convert the wavelength of the light outputted from the light emitting layers 8221 and output green light may be employed. The light emitter 822 is not necessarily configured to emit green light and may instead be configured to emit light having any other wavelength. For example, in the measurement of the blood oxygen level, infrared light and red light are used. In this case, a photoelectric sensor module capable of measuring the blood oxygen level can be configured, for example, by providing a filter layer that transmits the infrared light and the red light but blocks the other color light to control the light emitter to cause it to emit light having a desired color.

### Configuration of Light Emitter Terminals

The light emitter terminals 823 are provided in accordance with the light emitting areas of the light emitter 822, as shown in FIG. 6, and supplies the light emitting areas with electric power. In the present embodiment, since the light emitter 822 has the first light emitting area 822A and the second light emitting area 822B, the light emitter terminals 823 include the first light emitting area terminals 823A according to the first light emitting area 822A and the second light emitting area terminals 823B according to the second light emitting area 822B.

The first light emitting area terminals 823A are provided in a +Y-direction-side end portion of the silicon substrate 821 in accordance with the first light emitting area 822A. The first light emitting area terminals 823A include the terminal 8231, which is connected to the cathode layer 8222 in the first light emitting area 822A, and the terminal 8232, which is connected to the anode layer 8224 in the first light emitting area 822A. The first light emitting area terminals 823A are so configured that the terminal 8231 is shifted from the terminal 8232 toward the +X-direction side.

The second light emitting area terminals 823B are provided in a −Y-direction-side end portion of the silicon substrate 821 in accordance with the second light emitting area 822B. The second light emitting area terminals 823B include the terminal 8233, which is connected to the cathode layer 8222 in the second light emitting area 822B, and the terminal 8234, which is connected to the anode layer 8224 in the second light emitting area 822B. The second light emitting area terminals 823B are also so configured that the terminal 8233 is shifted from the terminal 8234 toward the +X-direction side.

The terminals 8231 to 8234 are so disposed in positions point-symmetric with respect to the light emitter 824 located at the center of the silicon substrate 821.

When electric power is supplied to the light emitter 822 via the light emitting area terminals 823A and 823B, the light emitter 822 emits light. In this process, the first light emitting area 822A and the first light emitting area terminals 823A are independent of the second light emitting area 822B and the second light emitting area terminals 823B. The first light emitting area 822A and the second light emitting area 822B can therefore emit light independently of each other.

### Configuration of Translucent Member

The translucent member 826 covers the +Z-direction side of the light emitter 822 and the light receiver 824 to protect the light emitter 822 and the light receiver 824, as shown in FIG. 7. The translucent member 826 is made of a translucent resin or glass material that transmits the light emitted from the light emitter 822 and the light outputted from the body of the user US and incident on the light receiver 824.

The thus configured translucent member 826 is exposed out of the housing 2 and can come into contact with the body of the user US after the photoelectric sensor module 8 is attached to the housing 2 and the light emitter 822 and the light receiver 824 are placed in the placement area 223 described above. That is, the translucent member 826 also serves as a contact member that comes into contact with the body of the user US.

### Configuration of Measurement Control Section

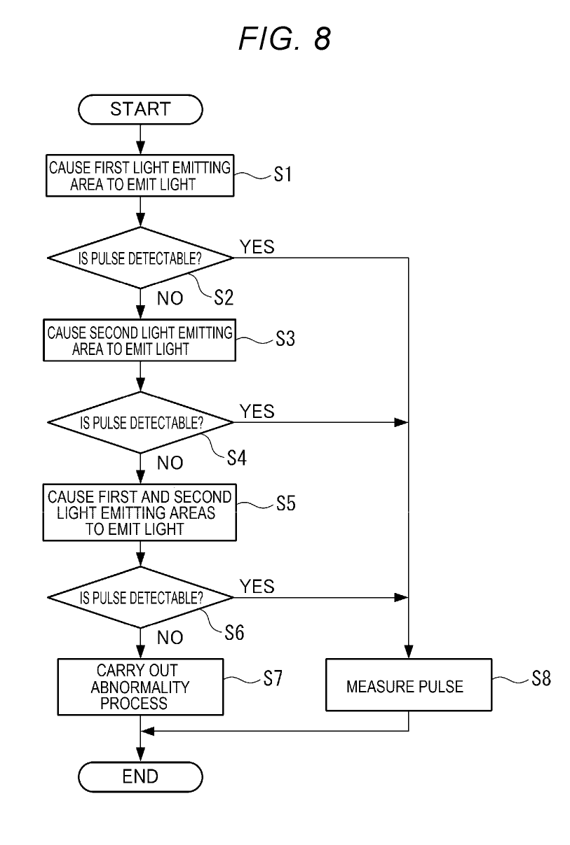

FIG. 8 is a flowchart showing the measurement control process carried out by the measurement controller 72.

The measurement controller 72 in the present embodiment carries out the following measurement control process when the photoelectric sensor module 8 is used to measure the pulse wave, which is the biological information, because the light emitter 822 of the photoelectric sensor module 8 has the plurality of light emitting areas (first light emitting area 822A and second light emitting area 822B). The measurement control process is carried out in accordance with a measurement control program stored in the storage 71.

In the measurement control process, the measurement controller 72 first causes the first light emitting area 822A to emit light (step S1), as shown in FIG. 8.

The measurement controller 72 then evaluates whether or not the pulse wave is detectable based on the pulse-wave signal inputted from the photoelectric sensor module 8 (step S2).

In a case where the measurement controller 72 determines in the evaluation process in step S2 that the pulse wave is detectable (YES in step S2), the measurement controller 72 proceeds to the process in step S8.

In a case where the measurement controller 72 determines in the evaluation process in step S2 that no pulse wave is detectable (NO in step S2), the measurement controller 72 causes the first light emitting area 822A to stop emitting light and causes the second light emitting area 822B to emit light (step S3).

After step S3, the measurement controller 72 evaluates whether or not the pulse wave is detectable based on the pulse-wave signal inputted from the photoelectric sensor module 8 (step S4).

In a case where the measurement controller 72 determines in the evaluation process in step S4 that the pulse wave is detectable (YES in step S4), the measurement controller 72 proceeds to the process in step S8.

In a case where the measurement controller 72 determines in the evaluation process in step S4 that no pulse wave is detectable (NO in step S4), the measurement controller 72 causes the first light emitting area 822A to emit light again so that the first light emitting area 822A and the second light emitting area 822B emit light (step S5).

After step S5, the measurement controller 72 evaluates whether or not the pulse wave is detectable based on the pulse-wave signal inputted from the photoelectric sensor module 8 (step S6).

In a case where the measurement controller 72 determines in the evaluation process in step S6 that the pulse wave is detectable (YES in step S6), the measurement controller 72 proceeds to the process in step S8.

In a case where the measurement controller 72 determines in the evaluation process in step S6 that no pulse wave is detectable (NO in step S6), the measurement controller 72 determines that abnormality has occurred in the pulse wave measurement and carries out an abnormality process (step S7). Specifically, the measurement controller 72 causes the notification section 5 to notify that abnormality has occurred in the pulse wave measurement. For example, the measurement controller 72 causes the display section 51 to display notification information representing that abnormality has occurred or the voice output section 52 to output predetermined voice.

After step S7, the measurement controller 72 terminates the measurement control process.

In step S8, the measurement controller 72 causes the photoelectric sensor module 8 to keep detecting the pulse wave and the analyzer 73 to analyze the inputted pulse-wave signal (step S8). After step S8, the measurement controller 72 terminates the measurement control process.

The measurement control process is carried out by the measurement controller 72 on a regular basis. The measurement control process may instead be carried out at the timing when the user US performs measurement control process input operation on the operation section 3.

### Steps of Manufacturing Photoelectric Sensor

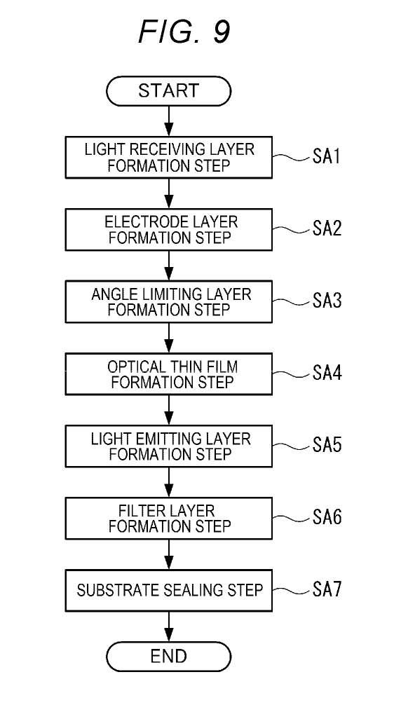

FIG. 9 is a flowchart showing the steps of manufacturing the photoelectric sensor 82A.

The photoelectric sensor 82A is manufactured, for example, by carrying out the manufacturing steps shown in FIG. 9.

In the manufacturing steps, steps SA1 to SA4 out of steps SA1 to SA7 are sequentially carried out to form the light receiver 824, which is the pn-junction-type photodiode, on the silicon substrate 821.

In step SA1, in which the light receiving layer is formed, an impurity is injected into a light receiver formation intended area of the silicon substrate 821 to form the light receiving layer 8241 (p-type area 8242 and n-type areas 8243).

In step SA2, in which the electrode layer is formed, the light receiving electrode layer 8244 is formed. Specifically, in the electrode layer formation step SA2, the p electrode layer 8245 and the n electrode layer 8246 are formed in accordance with the p-type area 8242 and the n-type areas 8243 of the light receiving layer 8241.

In the electrode layer formation step SA2, the light emitter terminals 823 and the light receiver terminals 825 are also formed.

In step SA3, in which the angle limiting layer is formed, the angle limiting layer 8247 is so formed as to cover the formed light receiving layer 8241 and light receiving electrode layer 8244.

In step SA4, in which the optical thin film layer is formed, the optical thin film layer 8248 is formed in a position shifted from the angle limiting layer 8247 in the direction +Z or a position on the angle limiting layer 8247 and according to the light receiver formation intended area.

The light receiver 824 is thus formed.

Steps SA5 and SA6 are then sequentially carried out on the silicon substrate 821 on which the light receiver 824 has been formed to form the light emitter 822, which is an organic EL device.

In step SA5, in which the light emitting layers are formed, the light emitting layers 8221 of the light emitter 822 is formed. Specifically, in the light emitting layer formation step SA5, the angle limiting layer 8247 formed in a light emitter formation intended area of the silicon substrate 821 is removed in an etching process. The cathode layers 8222, the organic layers 8223, and the anode layers 8224 are then sequentially formed.

In step SA6, in which the filter layers are formed, the filter layers 8225 are so formed as to cover the anode layers 8224. In the case where the light emitting layers 8221 can emit desired color light, the filter layer formation step SA6 can be omitted, as described above. Instead, in place of the filter layer formation step SA6, a wavelength conversion layer formation step of forming a wavelength conversion layer may be employed.

The light emitter 822 is thus formed.

After step SA6 described above, step SA7, in which the substrate is sealed, is carried out on the silicon substrate 821 on which the light emitter 822 and the light receiver 824 have been formed.

In the substrate sealing step SA7, the light emitter 822 and the light receiver 824 are sealed with a sealing resin, and the sealing resin is then planarized to form the translucent member 826.

The photoelectric sensor 82A is formed by carrying out the manufacturing steps described above.

### Effects Provided by First Embodiment

The biological information measurement apparatus 1 according to the present embodiment having been described above can provide the following effects.

The photoelectric sensor 82A includes the silicon substrate 821 and the light emitter 822, the light emitter terminals 823, the light receiver 824, and the light receiver terminals 825 provided on the surface 821A of the silicon substrate 821. The light emitter 822 and the light receiver 824 can therefore be configured in the form of a non-packaged bare chip on the surface 821A of the silicon substrate 821. The thickness of the photoelectric sensor 82A (not only dimension in direction in which light is emitted from light emitter 822 but dimension in direction +Z) can be reduced as compared with a case where a packaged LED chip and a packaged photodiode are mounted on the silicon substrate 821. Further, since the light emitter 822 and the light receiver 824 are configured on the silicon substrate 821, no packaged LED chip or photodiode needs to be attached to the silicon substrate 821, for example, via a bump. The dimension from the light emitter 822 and the light receiver 824 to the silicon substrate 821 can therefore be reduced. The thickness of the photoelectric sensor 82A can therefore be reduced, whereby a thinner photoelectric sensor 82A can be achieved. Moreover, since not only can the number of constituent parts provided in the photoelectric sensor 82A be reduced, but the steps of manufacturing the photoelectric sensor can be simplified, whereby the cost of the photoelectric sensor 82A can be reduced.

Further, since formation of the light emitter 822 and the light receiver 824 on the silicon substrate 821 eliminates the need to provide a gap for chip mounting between the light emitter 822 and the light receiver 824, the distance between the light emitter 822 and the light receiver 824 can be reduced. The amount of light received with the light receiver 824 can therefore be readily increased, whereby the sensitivity of the detection of the pulse wave detected with the photoelectric sensor 82A can be increased.

The light emitter 822, the light emitter terminals 823, the light receiver 824, and the light receiver terminals 825 are provided on the same surface 821A of the silicon substrate 821. The thickness of the photoelectric sensor 82A can therefore be further reduced as compared with a case where the light emitter 822 and the light receiver 824 are provided on the opposite surfaces of the silicon substrate 821, whereby a further thinner photoelectric sensor 82A can be achieved.

The light emitter 822 is an organic EL device. A light emitter 822 that can emit light can therefore be formed by formation of the cathode layers 8222, the organic layers 8223, and the anode layers 8224 on the silicon substrate 821. The light emitter 822 can therefore be readily formed on the silicon substrate 821.

The light emitter 822 has the first light emitting area 822A and the second light emitting area 822B, and the light emitter terminals 823 are provided in accordance with the light emitting areas 822A and 822B. That is, the light emitter terminals 823 include the first light emitting area terminals 823A according to the first light emitting area 822A and the second light emitting area terminals 823B according to the second light emitting area 822B. The light emitting areas 822A and 822B can therefore be separately caused to emit light, whereby the versatility of the photoelectric sensor 82A can be expanded.

The light emitter 822 is so disposed as to surround the light receiver 824. The light emitted from the light emitter 822 can therefore be readily incident on the light receiver 824 via the body of the user US. The amount of light received with the light receiver 824 can therefore be readily increased, whereby the sensitivity of the detection of the pulse wave detected with the photoelectric sensor 82A can be increased.

It is preferable that a photoelectric sensor that detects the pulse wave uses green light that has higher in-blood hemoglobin absorptance and is less affected by the ambient light than light having the other colors. In view of the fact described above, the light emitter 822 emits green light, whereby the accuracy of the detection of the pulse wave detected with the photoelectric sensor can be increased.

The light receiver 824 includes the angle limiting layer 8247, which limits the angle of incidence of the light incident on the light receiver 824. The angle limiting layer 8247 can prevent the light that is emitted from the light emitter 822 and does not travel via the body of the user US but externally travels toward the light receiver 824 or ambient light from being incident on the light receiver 824. False detection of the pulse wave can therefore be avoided, whereby the accuracy of the detection of the pulse wave detected with the photoelectric sensor 82A can be increased.

The light receiver 824 includes the optical thin film layer 8248, which transmits light that belongs to a wavelength band (predetermined band) to which the light emitted by the light emitter 822 belongs, out of the wavelength bands of the light incident on the light receiver 824, and prevents transmission of light that belongs to the other wavelength band. The optical thin film layer 8248 can therefore prevent the light receiver 824 from receiving light that belongs to a wavelength band different from the wavelength band to which the light that the light receiver 824 should receive belongs. The influence of noise can therefore be reduced, whereby the detection sensitivity of the photoelectric sensor 82A can be increased.

The photoelectric sensor 82A includes the translucent member 826, which covers the light emitter 822 and the light receiver 824. The translucent member 826 can prevent the light emitter 822 and the light receiver 824 from being directly exposed to the outside, whereby the light emitter 822 and the light receiver 824 can be protected.

The photoelectric sensor module 8 includes the photoelectric sensor 82A, the sensor control section 84, which controls the photoelectric sensor 82A, and the printed circuit board 81, on which the photoelectric sensor 82A and the sensor control section 84 are located. The sensor control section 84 controls the operation of turning on the light emitter 822 and processes the signal from the light receiver 824. Since the sensor control section 84 and the photoelectric sensor 82A are therefore located on the printed circuit board 81, the photoelectric sensor module 8 can have a compact configuration.

Since the sensor control section 84 has the function as the light emission controller and the function as the signal processor, the configuration of the photoelectric sensor module 8 can be simplified as compared with a case where circuits having the functions are separately provided.

The biological information measurement apparatus 1 includes the photoelectric sensor module 8, the circuit substrate CB, which derives the pulse rate, which is one piece of the biological information, based on the pulse-wave signal outputted from the photoelectric sensor module 8, and the connection member CM, which electrically connects the printed circuit board 81 in the photoelectric sensor module 8 to the circuit substrate CB. The primary control section 7, which is formed of the circuit substrate CB, can therefore reliably derive the pulse rate based on the result of the detection performed by the photoelectric sensor module 8. Since the photoelectric sensor module connected to the connection member CM can be changed to change biological information derivable by the circuit substrate CB, the versatility of the biological information measurement apparatus 1 can be increased.

### Second Embodiment

A second embodiment of the invention will next be described.

A biological information measurement apparatus according to the present embodiment has a configuration similar to that of the biological information measurement apparatus 1 shown in the first embodiment but differs from the biological information measurement apparatus 1 in that the photoelectric sensor further includes a light blocker disposed between the light emitter 822 and the light receiver 824. In the following description, the same or roughly the same portions as those having been already described have the same reference characters and will not be described.

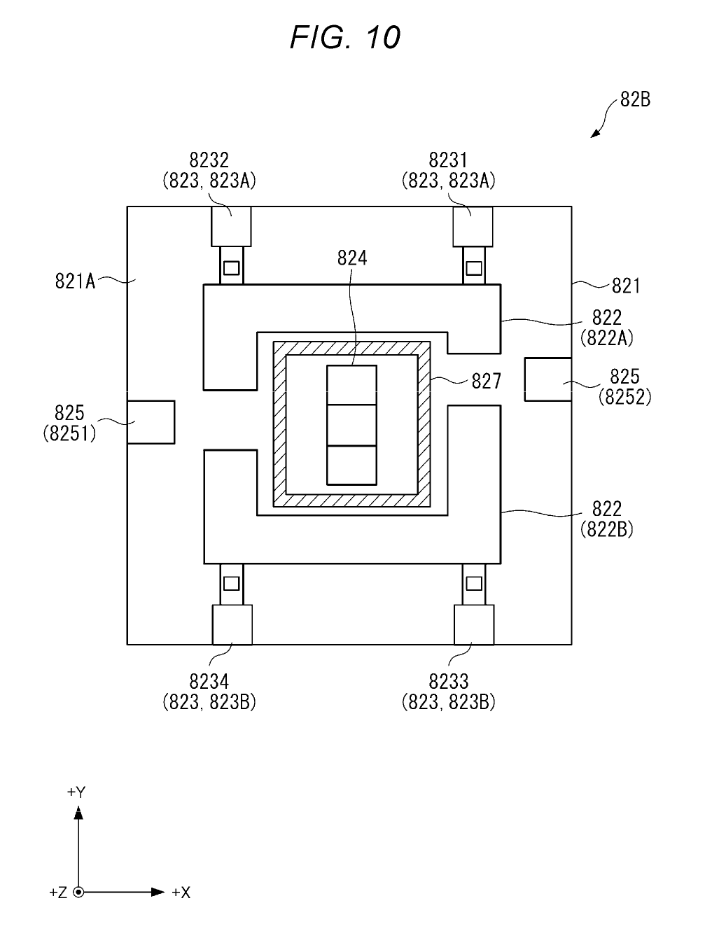

FIG. 10 shows a photoelectric sensor 82B provided in the biological information measurement apparatus according to the present embodiment viewed from the light output side (+Z-direction side). In FIG. 10, part of the configuration of the photoelectric sensor 82B is omitted.

The biological information measurement apparatus according to the present embodiment has the same configuration and function as those of the biological information measurement apparatus 1 except that the photoelectric sensor 82A is replaced with the photoelectric sensor 82B shown in FIG. 10. That is, the biological information measurement apparatus according to the present embodiment includes the photoelectric sensor module 8 including the photoelectric sensor 82B.

The photoelectric sensor 82B outputs light, receives light outputted from the body of the user US, detects the pulse wave, which is one piece of the biological information, and outputs the pulse-wave signal representing the detected pulse wave to the printed circuit board 81, as the photoelectric sensor 82A described above. The photoelectric sensor 82B includes a light blocker 827 in addition to the configuration of the photoelectric sensor 82A, as shown in FIG. 10. That is, the photoelectric sensor 82B includes the silicon substrate 821, the light emitter 822, the light emitter terminals 823, the light receiver 824, the light receiver terminals 825, the translucent member 826, and the light blocker 827.

The light blocker 827 prevents the light emitted from the light emitter 822 (first light emitting area 822A and second light emitting area 822B) from being directly incident on the light receiver 824 surrounded by the light emitter 822. In the present embodiment, the light blocker 827 is so formed on the surface 821A of the silicon substrate 821 as to be located in a position inside the light emitter 822 but outside the light receiver 824 when viewed in the direction +Z, which is the light output direction, and as to surround the light receiver 824. That is, the light blocker 827 is provided between the light emitter 822 and the light receiver 824.

Although not illustrated in detail, the thus configured light blocker 827 is so configured that a plurality of columnar elements that rise from the surface 821A of the silicon substrate 821 in the direction +Z are so arranged as to surround the light receiver 824. The columnar elements can be formed by layering a plurality of light blocking members on each other in a light blocking section formation intended area that is an area where the light blocker 827 is formed on the silicon substrate 821 in the electrode layer formation step SA2 and the angle limiting layer formation step SA3 out of the manufacturing steps described above. That is, the light blocker 827 can be formed on the silicon substrate 821 along with the light emitter 822 and the light receiver 824 when the photoelectric sensor 82B is manufactured.

### Effects Provided by Second Embodiment

The biological information measurement apparatus according to the present embodiment described above can provide not only the same effects as those provided by the biological information measurement apparatus 1 described above but the following effects.

The photoelectric sensor 82B includes the light blocker 827, which is provided between the light emitter 822 and the light receiver 824 and prevents the light emitted from the light emitter 822 from being directly incident on the light receiver 824. The accuracy of the detection of the pulse wave detected with the photoelectric sensor 82B can therefore be increased.

Further, since the light blocker 827 is formed on the silicon substrate 821, separately attaching a member that forms the light blocker to the photoelectric sensor 82A and other efforts can be omitted.

The distance between the light emitter and the light receiver is preferably small as described above. To separately attach a light blocker between the light emitter and the light receiver, however, a small distance between the light emitter and the light receiver requires troublesome attachment work. In contrast, since the light blocker 827 is formed on the silicon substrate 821, the step of forming the light blocker 827 can be part of the step of forming the light emitter 822 and the light receiver 824 on the silicon substrate 821. The light blocker can therefore be readily formed between the light emitter 822 and the light receiver 824 even when the distance between the light emitter 822 and the light receiver 824 is small. A photoelectric sensor 82B that allows improvement in the pulse wave detection accuracy can therefore be readily manufactured.

### Variations of Embodiments

The invention is not limited to the embodiments described above, and variations, improvements, and other modifications to the extent that the advantage of the invention is achieved fall within the scope of the invention.

### Variation of Number of Light Emitting Areas

In the embodiments described above, the photoelectric sensors 82A and 82B each include the light emitter 822, which has the two light emitting areas 822A and 822B, and the one light receiver 824, but not necessarily. The photoelectric sensors 82A and 82B may each be replaced with a photoelectric sensor having a different number of light emitting areas that differs from the number in the photoelectric sensors 82A and 82B.

### First Variation

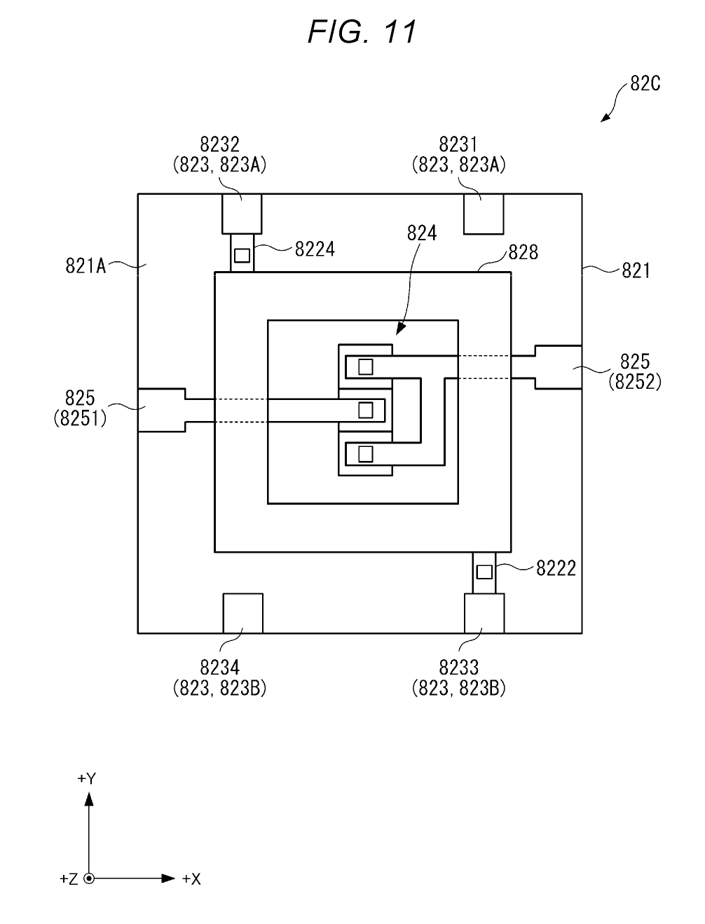

FIG. 11 shows a photoelectric sensor 82C, which is a first variation of the photoelectric sensor 82A, viewed from the light output side.

For example, the photoelectric sensor 82C shown in FIG. 11 may be employed in place of the photoelectric sensors 82A and 82B.

The photoelectric sensor 82C has the same configuration and function as those of the photoelectric sensor 82A except that the light emitter 822 is replaced with a light emitter 828, as shown in FIG. 11. That is, the photoelectric sensor 82C includes the silicon substrate 821, the light emitter 828, the light emitter terminals 823, the light receiver 824, and the light receiver terminals 825, which are provided on the surface 821A of the silicon substrate 821, and the translucent member 826.

The light emitter 828 is formed in a rectangular frame-like shape that surrounds the light receiver 824, has one light emitting area that emits green light, and has the same layer structure as that of the light emitter 822. That is, the light emitter 828 is an organic EL device having the light emitting layer 8221 and the filter layer 8225 described above.

Out of the cathode layer 8222 and the anode layer 8224, which form the light emitting layer 8221 of the light emitter 828, the cathode layer 8222 is connected to any of the terminals 8231 to 8234, which form the light emitter terminals 823. The anode layer 8224 is connected to the other terminals excluding the terminal connected to the cathode layer 8222 out of the terminals 8231 to 8234. In the example shown in FIG. 11, the cathode layer 8222 is connected to the terminal 8233, and the anode layer 8224 is connected to the terminal 8232.

Also in the case where the thus configured photoelectric sensor 82C is employed in place of the photoelectric sensor 82A, the same effects as those shown in the first embodiment can be provided. In a case where the photoelectric sensor 82C includes the light blocker 827 disposed between the light emitter 828 and the light receiver 824, the same effects as those shown in the second embodiment can be provided.

In the case of the measurement apparatus 1 including the photoelectric sensor 82B, in which the photoelectric sensor 82B has one light emitting area, steps S3 to S6 in the measurement control process described above are omitted.

### Second Variation

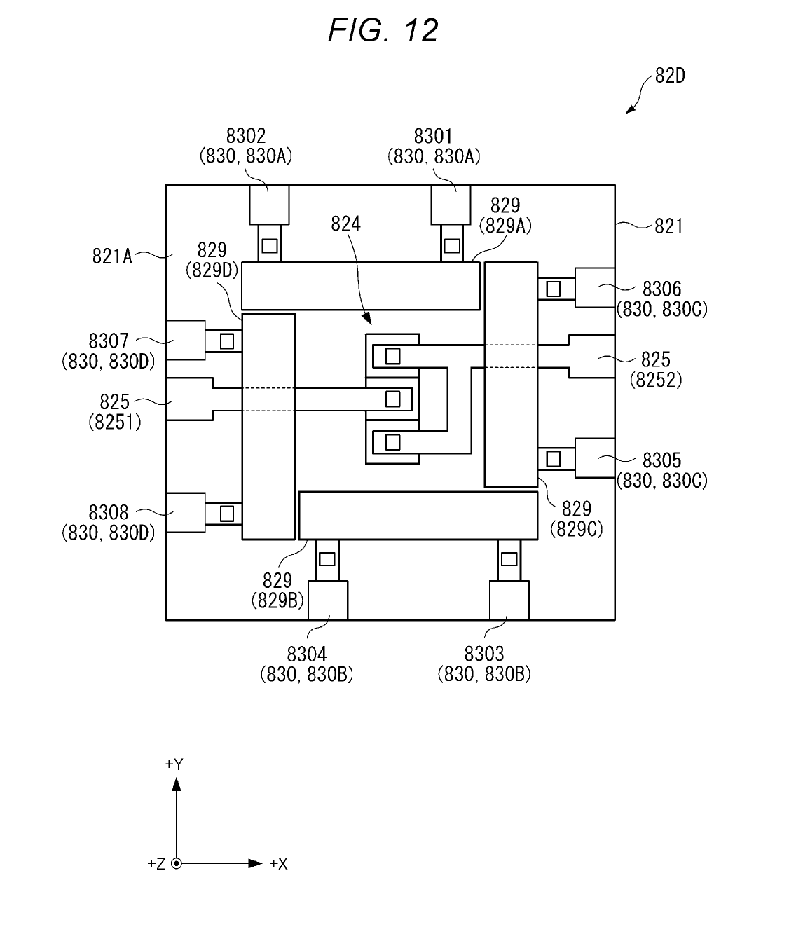

FIG. 12 shows a photoelectric sensor 82D, which is a second variation of the photoelectric sensor 82A, viewed from the light output side.

For example, the photoelectric sensor 82D shown in FIG. 12 may be employed in place of the photoelectric sensors 82A and 82B.

The photoelectric sensor 82D has the same configuration and function as those of the photoelectric sensor 82A except that the light emitter 822 and the light emitter terminals 823 are replaced with a light emitter 829 and light emitter terminals 830, as shown in FIG. 12. That is, the photoelectric sensor 82D includes the silicon substrate 821, the light emitter 829, the light emitter terminals 830, the light receiver 824, and the light receiver terminals 825, which are provided on the surface 821A of the silicon substrate 821, and the translucent member 826.

The light emitter 829 has four light emitting areas 829A to 829D, which emit green light and are divided areas, and the four light emitting areas 829A to 829D are so disposed as to form a rectangular frame-like shape that surrounds the light receiver 824 when viewed from the +Z-direction side. In detail, the first light emitting area 829A is located on the +Y-direction side of the light receiver 824. The second light emitting area 829B is located on the −Y-direction side of the light receiver 824. The third light emitting area 829C is located on the +X-direction side of the light receiver 824. The fourth light emitting area 829D is located on the −X-direction side of the light receiver 824.

The thus configured light emitter 829 is an organic EL device having the same layer structure as that of the light emitter 822 and is formed on the surface 821A. That is, the light emitting areas 829A to 829D each have the light emitting layer 8221 and the filter layer 8225.

The light emitter terminals 830 function in the same manner in which the light emitter terminals 823 function. The light emitter terminals 830 include a set of terminals connected to the cathode layers 8222 and a set of terminals connected to the anode layers 8224 in accordance with the light emitting areas 829A to 829D. That is, the light emitter terminals 830 include first light emitting area terminals 830A according to the first light emitting area 829A, second light emitting area terminals 830B according to the second light emitting area 829B, third light emitting area terminals 830C according to the third light emitting area 829C, and fourth light emitting area terminals 830D according to the fourth light emitting area 829D, and the light emitting area terminals 830A to 830D are formed on the surface 821A.

The first light emitting area terminals 830A are provided in a +Y-direction-side end portion of the silicon substrate 821 in accordance with the first light emitting area 829A. The first light emitting area terminals 830A includes a terminal 8301, which is connected to the cathode layer 8222 in the first light emitting area 829A, and a terminal 8302, which is connected to the anode layer 8224 in the first light emitting area 829A.

The second light emitting area terminals 830B are provided in a −Y-direction-side end portion of the silicon substrate 821 in accordance with the second light emitting area 829B. The second light emitting area terminals 830B includes a terminal 8303, which is connected to the cathode layer 8222 in the second light emitting area 829B, and a terminal 8304, which is connected to the anode layer 8224 in the second light emitting area 829B.

The third light emitting area terminals 830C are provided in a +X-direction-side end portion of the silicon substrate 821 in accordance with the third light emitting area 829C. The third light emitting area terminals 830C includes a terminal 8305, which is connected to the cathode layer 8222 in the third light emitting area 829C, and a terminal 8306, which is connected to the anode layer 8224 in the third light emitting area 829C.

The fourth light emitting area terminals 830D are provided in a −X-direction-side end portion of the silicon substrate 821 in accordance with the fourth light emitting area 829D. The fourth light emitting area terminals 830D includes a terminal 8307, which is connected to the cathode layer 8222 in the fourth light emitting area 829D, and a terminal 8308, which is connected to the anode layer 8224 in the fourth light emitting area 829D.

The light emitting area terminals 830A to 830D are connected, although not shown, to the substrate-side electrodes 812 on the printed circuit board 81 via the FPCs 813. The light emitting areas 829A to 829D are caused to emit light and stop emitting light by the sensor control section 84 under the control of the primary control section 7.

Also in the case where the thus configured photoelectric sensor 82D is employed in place of the photoelectric sensor 82A, the same effects as those shown in the first embodiment can be provided. In a case where the photoelectric sensor 82D includes the light blocker 827, the same effects as those shown in the second embodiment can be provided.

In the measurement apparatus 1 including the photoelectric sensor 82D, the measurement controller 72 sequentially causes each of the light emitting areas 829A to 829D to emit light and evaluates the detection of the pulse wave in the measurement control process described above (processes carried out in steps S1 and S2, for example) to measure the pulse wave based on an appropriate pulse wave detectable combination of a light emitting area and the light receiver.

### Other Variations

In the embodiments and variations described above, the light emitters 822, 828, and 829 and the light receiver 824 are provided on the surface 821A, which is oriented in the direction +Z, of the silicon substrate 821. The surface 821A is not necessarily flat and may have a step. That is, on the silicon substrate, the portion where the light emitter is located and the portion where the light receiver is located may differ from each other in terms of position in the direction +Z. Further, the light emitter terminals and the light receiver terminals may be provided on a surface of the silicon substrate different from the surface on which the light emitter and the light receiver are provided. Even in the case where the light emitter and the light receiver are formed on the same surface, the positions of the light emitter terminals and the light receiver terminals can be changed as appropriate. That is, the light emitting surface of the light emitter and the light receiving surface of the light receiver only need to be oriented in the same direction.

On the other hand, on the silicon substrate, the surface on which the light emitter is located and the surface on which the light receiver is located may differ from each other. For example, on the silicon substrate, the light receiver may be provided on the surface opposite the surface on which the light emitter is provided.

In the embodiments and variations described above, the light emitters 822, 828, and 829 each have alight emitting area formed of an organic EL device, but not necessarily. A light emitter having a light emitting area formed of a light emitting device of another type may be formed as long as the light emitter can be formed on the silicon substrate.

In the embodiments and variations described above, the light emitters 822, 828, and 829 have the two, one, and four light emitting areas, respectively, but not necessarily. The number of light emitting areas of the light emitter can be changed as appropriate.

In the embodiments and variations described above, the light emitters 822, 828, and 829 are each so disposed as to surround the light receiver 824, but not necessarily. The position where the light emitter is disposed relative to the light receiver can be changed as appropriate, and the light emitter is not necessarily so disposed as to surround the light receiver. For example, one light emitter and one light receiver may be disposed side by side along the direction +X or +Y. Instead, for example, one light receiver may be disposed between two light emitters, or one light emitter may be disposed between two light receivers. Still instead, two light receivers may be so disposed as to be adjacent to one light emitter. In this case, one of the two light receivers may be disposed between the other light receiver and the one light emitter, or the two light receivers may be disposed side by side in one of the directions +X and +Y and the one light emitter may be disposed next to the two light receivers in the other one of the directions +X and +Y.

In the embodiments and variations described above, in which the photoelectric sensors 82A to 82D each detect the pulse wave, the light emitters 822, 828, and 829 each emit green light, but not necessarily. The color light emitted by the light emitter can be changed as appropriate in accordance with a detection target to be detected with the photoelectric sensor. For example, the light emitter may emit infrared light or blue light.

In the embodiments and variations described above, the light receiver 824 includes the angle limiting layer 8247, but not necessarily, and the angle limiting layer 8247 may be omitted. For example, the light receiver 824 may include the light receiving layer 8241 and the optical thin film layer 8248 but may not include the angle limiting layer 8247, or the light receiver 824 may include the light receiving layer 8241 but may not include the angle limiting layer 8247 or the optical thin film layer 8248.

In the embodiments and variations described above, the photoelectric sensors 82A to 82D each include the translucent member 826, which covers the light emitters 822, 828, and 829 and the light receiver 824, but not necessarily, and the translucent member 826 may be omitted. Instead, another translucent member fit into or otherwise placed in the opening 222 described above may be so provided as to cover the +Z-direction side of the light emitter and the light receiver placed in the placement section 223.

In the embodiments and variations described above, the photoelectric sensor module 8 is so configured that the sensor control section 84, which controls the photoelectric sensors 82A to 82D, is disposed on the printed circuit board 81, on which the photoelectric sensors 82A to 82D are disposed, but not necessarily, and the sensor control section 84 may be disposed on the circuit substrate CB or in any other portion.

The sensor control section 84 has the function of the turn-on controller and the function of the signal processor, but not necessarily, and the sensor control section may have only one of the functions.

In the embodiments and variations described above, the circuit substrate CB, which forms the primary control section 7, and the photoelectric sensor module 8 are electrically connected to each other via the connection member CM, but not necessarily, and the photoelectric sensor module may be provided with a circuit having the function of the analyzer 73, or the primary control section 7 may be provided with the photoelectric sensor module 8. The photoelectric sensor module 8 and the circuit substrate CB may instead wirelessly communicate with each other.

In the embodiments and variations described above, the light emitting areas of the light emitters 822, 828, and 829 have the same filter layer 8225, but not necessarily. In the case where any of the light emitters has a plurality of light emitting areas, at least one of the light emitting areas may be provided a filter layer or a polarizing layer different from the filter layers or polarizing layers in the other light emitting areas to cause the type of the light emitted from the light emitter to vary in accordance with the application of the photoelectric sensor. Further, the number of light emitting areas to be caused to emit light may be adjusted in accordance with the state of detection of a detection target, the environment around the detection target, or any other factor to adjust the amount of light emitted from the light emitter.

In the embodiments and variations described above, the photoelectric sensors 82A to 82D each detect the pulse wave as the biological information, the photoelectric sensor module 8 including any of the photoelectric sensors 82A to 82D outputs the pulse-wave signal representing the pulse wave to the primary control section 7, and the analyzer 73 of the primary control section 7 analyzes the pulse rate based on the inputted pulse-wave signal. That is, the biological information measurement apparatus 1 measures the pulse wave and the pulse rate, which are each the biological information, but not necessarily, and the biological information that can be measured by the biological information measurement apparatus according the embodiment of the invention is not limited the pulse wave and the pulse rate. For example, the biological information measurement apparatus may measure other pieces of biological information, such as the heart rate variability (HRV), R-R interval (RRI: interval between pulses), blood pressure, blood sugar level, amount of activity, consumed calorie, and maximum oxygen uptake (VO₂max), based on the result of the detection performed by any of the photoelectric sensors having the configurations described above.

The biological information measurement apparatus 1 may further include an acceleration sensor, a gyro sensor, or any other motion sensor that can detect the user's body motion information, an orientation sensor that detects the orientation, a position sensor that can measure position information (GPS sensor, for example), or any other sensor.

What is claimed is:

1. A photoelectric sensor comprising:
a silicon substrate;
a light emitter that emits first light and is provided on the silicon substrate;
a light receiver that receives second light and is provided on the silicon substrate;
a light emitter terminal that supplies the light emitter with electric power and is provided on the silicon substrate; and
a light receiver terminal to which the light receiver outputs a signal and is provided on the silicon substrate.

2. The photoelectric sensor according to claim 1,
wherein the light emitter and the light receiver are provided on the same surface of the silicon substrate.

3. The photoelectric sensor according to claim 1,
further comprising a light blocker provided on the silicon substrate and between the light emitter and the light receiver.

4. The photoelectric sensor according to claim 1,
wherein the light emitter is an organic EL device.

5. The photoelectric sensor according to claim 1,
wherein the light emitter has a plurality of light emitting areas, and
the light emitter terminal is provided in accordance with the plurality of light emitting areas.

6. The photoelectric sensor according to claim 1,
wherein the light emitter is so disposed as to surround the light receiver.

7. The photoelectric sensor according to claim 1,
wherein the light emitter emits green light.

8. The photoelectric sensor according to claim 1,
wherein the light receiver includes an angle limiting layer that limits an angle of incidence of the light incident on the light receiver.

9. The photoelectric sensor according to claim 1,
wherein the light receiver includes an optical thin film layer that transmits light that belongs to a predetermined band out of wavelength bands of light incident on the light receiver.

10. The photoelectric sensor according to claim 1,
further comprising a translucent member that covers the light emitter and the light receiver.

11. A photoelectric sensor module comprising:
the photoelectric sensor according to claim 1;
a sensor control section that controls the photoelectric sensor; and
a printed circuit board on which the photoelectric sensor and the sensor control section are located,
wherein the sensor control section at least turns on the light emitter and/or processes the signal from the light receiver.

12. A photoelectric sensor module comprising:
the photoelectric sensor according to claim 2;
a sensor control section that controls the photoelectric sensor; and
a printed circuit board on which the photoelectric sensor and the sensor control section are located,
wherein the sensor control section at least turns on the light emitter and/or processes the signal from the light receiver.

13. A photoelectric sensor module comprising:
the photoelectric sensor according to claim 3;
a sensor control section that controls the photoelectric sensor; and
a printed circuit board on which the photoelectric sensor and the sensor control section are located,
wherein the sensor control section at least turns on the light emitter and/or processes the signal from the light receiver.

14. A photoelectric sensor module comprising:
the photoelectric sensor according to claim 4;
a sensor control section that controls the photoelectric sensor; and
a printed circuit board on which the photoelectric sensor and the sensor control section are located,
wherein the sensor control section at least turns on the light emitter and/or processes the signal from the light receiver.

15. A photoelectric sensor module comprising:
the photoelectric sensor according to claim 5;
a sensor control section that controls the photoelectric sensor; and
a printed circuit board on which the photoelectric sensor and the sensor control section are located,
wherein the sensor control section at least turns on the light emitter and/or processes the signal from the light receiver.

16. A photoelectric sensor module comprising:
the photoelectric sensor according to claim 6;
a sensor control section that controls the photoelectric sensor; and
a printed circuit board on which the photoelectric sensor and the sensor control section are located,
wherein the sensor control section at least turns on the light emitter and/or processes the signal from the light receiver.

17. A photoelectric sensor module comprising:
the photoelectric sensor according to claim 7;
a sensor control section that controls the photoelectric sensor; and
a printed circuit board on which the photoelectric sensor and the sensor control section are located,
wherein the sensor control section at least turns on the light emitter and/or processes the signal from the light receiver.

18. A photoelectric sensor module comprising:
the photoelectric sensor according to claim 8;
a sensor control section that controls the photoelectric sensor; and
a printed circuit board on which the photoelectric sensor and the sensor control section are located,
wherein the sensor control section at least turns on the light emitter and/or processes the signal from the light receiver.

19. A photoelectric sensor module comprising:
the photoelectric sensor according to claim 9;
a sensor control section that controls the photoelectric sensor; and
a printed circuit board on which the photoelectric sensor and the sensor control section are located,
wherein the sensor control section at least turns on the light emitter and/or processes the signal from the light receiver.

20. A biological information measurement apparatus comprising:
the photoelectric sensor module according to claim 11;
a circuit substrate that derives biological information based on a signal outputted from the photoelectric sensor module; and
a connection member that electrically connects the printed circuit board to the circuit substrate.
